(19) United States
(12) Patent Application Publication
(10) Pub. No.: US 2021/0137403 A1
(43) Pub. Date: May 13, 2021

(54) SENSORY SUBSTITUTION CONTROL DEVICE AND METHOD

(71) Applicant: Electronics and Telecommunications Research Institute, Daejeon (KR)

(72) Inventors: Moo Seop KIM, Sejong-si (KR); Kyeong Deok MOON, Daejeon (KR); Yun Kyung PARK, Daejeon (KR); Chi Yoon JEONG, Daejeon (KR)

(73) Assignee: Electronics and Telecommunications Research Institute, Daejeon (KR)

(21) Appl. No.: 17/091,575

(22) Filed: Nov. 6, 2020

(30) Foreign Application Priority Data
Nov. 8, 2019 (KR) 10-2019-0142452
Oct. 5, 2020 (KR) 10-2020-0128249

(51) Int. Cl. A61B 5/0484; A61B 5/04

(52) U.S. Cl. CPC A61B 5/04842; A61B 2560/0242; A61B 5/04012

(57) ABSTRACT

Provided are a sensory substitution control device and method. The sensory substitution device includes a reaction signal collector configured to collect a user's response or reaction signal to a sensory substitution signal received from a sensory substitution device, an environmental information collector configured to collect environmental information of the user who receives the substitution signal, a sensory substitution efficiency analyzer configured to analyze the user's sensory substitution efficiency delivered by the sensory substitution device on the basis of the collected environmental information and generate an adjustment signal for adjusting the substitution signal according to the sensory substitution efficiency, and an adjustment signal transmitter configured to provide a correction signal according to the analyzed environmental information-specific sensory substitution efficiency of the sensory substitution signal to the sensory substitution device.

### CROSS-REFERENCE TO RELATED APPLICATION

This application claims priority to and the benefit of Korean Patent Application No. 10-2019-0142452, filed on Nov. 8, 2019, and Korean Patent Application No. 10-2020-0128249, filed on Oct. 5, 2020, the disclosure of which is incorporated herein by reference in its entirety.

### BACKGROUND

### 1. Field of the Invention

The present invention relates to a sensory substitution control device and method for combining and converting two or more pieces of multisensory information into another piece of multisensory information and, more particularly, relates to a sensory substitution control device and method for efficiently transmitting multisensory information by recombining multisensory information, which is input according to a user's physical sensory characteristics, into a plurality of pieces of target sense information.

### 2. Discussion of Related Art

With an increase in the average lifespan based on the development of medical technology, the number of people, who suffer from degradation in functions, such as vision and hearing, due to aging or have a damaged sense due to an accident or disease and thus are disabled, is increasing. Accordingly, sensory substitution technology is attracting attention as a technical measure for enabling the people to continuously perform economic activities and for improving their quality of life by improving their damaged senses and perception abilities.

In general, sensory substitution means a change of information on a sense which is functionally damaged or degraded into another sensory modality signal. Such sensory substitution technology is known to be based on brain plasticity which refers to the human brain's ability to be structurally and functionally modified and reorganized to adapt to a new environment. When a sensory ability of a person is degraded or lost, regions of the cerebral cortex taking charge of sense information are rearranged. In the case of functional loss of a specific sense in the human body, such development allows another sense to replace the function and adapt to the situation.

Sensory substitution devices or methods according to related arts convert a specific single sensory signal of human senses into another sensory signal and transmit the sensory signal. In some technologies, a plurality of sensory organs are used as a transmission path in order to transmit a single sensory signal.

However, most sensory substitution devices are limited to methods or devices for converting a signal. In general, a user who receives a sensory substitution signal may prefer a sense varying according to a sensory organ which is a sensory receptor and may react to the received sensory substitution signal to different degrees. Consequently, when a sensory substitution signal is converted without reflecting characteristics of a user who receives the sensory substitution signal, it is difficult to efficiently transfer information.

### SUMMARY OF THE INVENTION

The present invention is directed to providing a sensory substitution control device that allows adjustment of a substitution signal provided by a sensory substitution device on the basis of a response or reaction of a user who receives the substitution signal for replacing a sense from the sensory substitution device.

The present invention is directed to providing a sensory substitution control device that allows efficient substitution of a user's sense by adjusting a substitution signal according to the user's characteristics in a sensory substitution process employing a sensory substitution device.

Objects of the present invention are not limited to those mentioned above, and other objects which have not been mentioned will be clearly understood by those of ordinary skill in the art from the following description.

According to an aspect of the present invention, there is provided a sensory substitution control device including a reaction signal collector configured to collect a user's response or reaction signal to a sensory substitution signal received from a sensory substitution device, an environmental information collector configured to collect environmental information of the user who receives the substitution signal, a sensory substitution efficiency analyzer configured to analyze the user's sensory substitution efficiency delivered by the sensory substitution device on the basis of the collected environmental information and generate an adjustment signal for adjusting the substitution signal according to the sensory substitution efficiency, and an adjustment signal transmitter configured to provide a correction signal according to the analyzed environmental information-specific sensory substitution efficiency of the sensory substitution signal to the sensory substitution device.

The substitution signal may include at least one of a video signal and an audio signal, and the environmental information may be one of brightness information, indoor or outdoor location information, and sealing information.

As the reaction signal collector, one of the following may be used: a brainwave monitor for measuring brainwaves or electroencephalogram (EEG) signals and magnetic resonance imaging (MRI) equipment for collecting reactions or activities of a changed brain function and structure as brain image information by measuring the brain of the user who receives the substitution signal through continuous learning of sensory substitution signals.

The reaction signal collector may collect the reaction signal varying according to the degree of learning and perception.

The reaction signal collector may use an invasive method of collecting data directly from the brain.

The reaction signal collector may use a non-invasive method of using the brain surface or equipment.

The sensory substitution efficiency analyzer may analyze the reactivity of a brain region taking charge of a substitutive sense, which is replaced by the sensory substitution device, with respect to the user who receives the substitution signal and may determine the sensory substitution efficiency to be higher when the reactivity is higher or may determine the sensory substitution efficiency to be lower when the reactivity is lower.

### BRIEF DESCRIPTION OF THE DRAWINGS

The above and other objects, features and advantages of the present invention will become more apparent to those of ordinary skill in the art by describing exemplary embodiments thereof in detail with reference to the accompanying drawings, in which:

FIG. 1 is a block diagram of a sensory substitution control device according to an exemplary embodiment of the present invention;

FIG. 2 is a diagram illustrating a configuration of a sensory substitution system to which the sensory substitution control device of FIG. 1 is applied;

FIG. 3 is an exemplary conceptual diagram of a sensory substitution control process of FIG. 1;

FIG. 4 is a block diagram illustrating a detailed configuration of a sensory substitution efficiency analyzer of FIG. 1;

FIG. 5 is a flowchart illustrating a method of analyzing sensory substitution efficiency in sensory substitution control according to the exemplary embodiment of the present invention; and

FIG. 6 is a flowchart illustrating a method of transmitting an adjustment signal to a sensory substitution device in FIG. 5.

### DETAILED DESCRIPTION OF EXEMPLARY EMBODIMENTS

Advantages and features of the present invention and methods for achieving them will be made clear from exemplary embodiments described below in detail with reference to the accompanying drawings. However, the present invention is not limited to the exemplary embodiments disclosed below and can be embodied in various different forms. The embodiments are provided so that this disclosure of the present invention will be thorough and complete and will fully convey the scope of the invention to those of ordinary skill in the art. The present invention is only defined by the claims. Meanwhile, terminology used in this specification is for the purpose of describing the embodiments and is not intended to limit the present invention. In this specification, the singular forms include the plural forms as well unless the context clearly indicates otherwise. The terms “comprise” and/or “comprising,” when used herein, do not preclude the presence or addition of one or more elements, steps, operations, and/or devices other than stated elements, steps, operations, and/or devices.

FIG. 1 is a block diagram illustrating a sensory substitution control device according to an exemplary embodiment of the present invention.

As shown in FIG. 1, the sensory substitution control device according to the exemplary embodiment of the present invention includes a reaction signal collector 110, an environmental information collector 120, a sensory substitution efficiency analyzer 130, and an adjustment signal transmitter 140.

The reaction signal collector 110 collects a user's response or reaction signal to a sensory substitution signal received from a sensory substitution device 200. The substitution signal includes one or more video signals or audio signals.

In the exemplary embodiment of the present invention, the reaction signal collector 110 collects a reaction signal from the sensory substitution device 200 including a brainwave monitor for measuring brainwaves or electroencephalogram (EEG) signals and magnetic resonance imaging (MRI) equipment for collecting reactions or activities of a changed brain function and structure as brain image information by measuring the brain of the user who receives the substitution signal through continuous learning of sensory substitution signals.

For example, a brainwave monitor for measuring brainwaves or EEG signals may be used to collect the user's response or reaction to the sensory substitution signal. A signal change of the central occipital channel (Oz) and the right lateral parieto-occipital lobe (PO8) is used to measure a reaction to visual information, and a signal change of the right lateral parieto-temporal lobe (TP8) and the right lateral frontal lobe (F8) is used to measure a reaction to auditory information.

To measure changes in a brain function and a brain structure, MRI equipment may be used. The lateral occipital tactile-visual (LOtv) region may be used for measured visual cortex (VC), auditory cortex (AC), and visual and tactile information, and the reactivity of the secondary somatosensory regions and the like may be used for the tactile sensation and temperature.

The reaction signal collector 110 may comprehensively use all of a method of collecting reaction signals according to the degrees of learning and perception, an invasive method of collecting data directly from the brain, and a non-invasive method of using the brain surface or equipment.

The environmental information collector 120 collects environmental information of the user who receives the substitution signal. The environmental information includes one of brightness information, indoor or outdoor location information, and sealing information. When multisensory information is transmitted to the user, sensory suppression may occur, and a suppressed sense may vary according to the user's action (movement, stopping, etc.). Therefore, the environmental information collector 120 may collect and use brightness information (Eb) or location information (Ep) of surroundings of the user who receives the sensory substitution signal or action information (Ea) of the user.

The sensory substitution efficiency analyzer 130 analyzes sensory substitution efficiency of each piece of the collected environmental information on the basis of a user-reflecting signal based on the substitution signal.

FIG. 4 is a block diagram illustrating a detailed configuration of the sensory substitution efficiency analyzer 130 according to the exemplary embodiment of the present invention.

As shown in FIG. 4, the sensory substitution efficiency analyzer 130 includes a reaction signal extractor 131, a reactivity calculator 132, a brain action data extractor 133, a brain activity calculator 134, a learning part 135, and a substitutive sense determiner 136.

The reaction signal extractor 131 extracts a reaction signal to a brainwave signal (a visual information reaction signal), which is provided by a brainwave signal collector of the reaction signal collector 110, through a visual information reaction signal extractor including a central occipital signal extractor and a right lateral parieto-occipital lobe signal extractor.

The reaction signal extractor 131 extracts an auditory information reaction signal through an auditory information reaction signal extractor including a right lateral parieto-temporal lobe signal extractor and a right lateral parieto-frontal lobe signal extractor and extracts a tactile information reaction signal through a tactile information reaction signal extractor including a cerebral vertex signal extractor.

The reactivity calculator 132 calculates variations through a visual information reaction variation calculator, an auditory information reaction variation calculator, and a tactile information reaction variation calculator on the basis of base information stored in a base information storage according to the type of a signal extracted by the reaction signal extractor 131. In other words, the reactivity calculator 132 calculates the reaction variation of each individual sensory signal, and calculation of the reaction variation of each piece of individual sense information may involve a frequency converter for analysis, a delta and theta wave extractor, and an individual signal reaction variation calculator.

For example, even when a visual stimulus is given to the sensory substitution device 200, a visual information reaction signal, an auditory information reaction signal, and a tactile information reaction signal are input through the reaction signal extractor 131 so that the degrees of visual, auditory, and tactile reactions to visual information may be calculated. After the frequency of each signal is converted through the visual information reaction variation calculator, the auditory information reaction variation calculator, and the tactile information reaction variation calculator, a delta wave and a theta wave are extracted and compared with the base information so that reaction variations may be calculated. The frequency conversion may employ Fourier transform or fast Fourier transform.

As criteria for high and low reactivity, a high-low value (e.g., double or more) and a threshold value (e.g., 40%) of reactivity may be set on the basis of the base information. When the reactivity is greater than the high-low value, the reactivity may be determined to be high. When the reactivity is greater than the base information by the threshold value or more, the reaction may be determined to be related to a stimulus. When the reactivity is smaller than the base information by the threshold value or more, the reactivity may be determined to be low.

For the reactivity analysis, frequency analysis of brainwave signals, the event-related potential (ERP) technique, the event-related spectral perturbation (ERSP) method, etc. may be used.

The brain action data extractor 133 extracts visual cortex brain action data, auditory cortex brain action data, and somatosensory cortex brain action data from data collected through a brain action data collector of the reaction signal collector 110.

The brain activity calculator 134 includes a visual cortex activity calculator, an auditory cortex activity calculator, and a somatosensory cortex activity calculator which calculate a visual cortex activity, an auditory cortex activity, and a somatosensory cortex activity using the visual cortex brain action data, the auditory cortex brain action data, and the somatosensory cortex brain action data extracted by the brain action data extractor 133, respectively. By means of such a configuration, the brain activity calculator 134 calculates the visual cortex activity, the auditory cortex activity, and the somatosensory cortex activity.

The learning part 135 explicitly represents the priority order of sense information preferred by the user and whether reactivity is high or low as learning progresses for each individual sense.

The substitutive sense determiner 136 calculates the sensory substitution efficiency (S=R+A+E) of each individual substitutive sense using the reactivity (R), the activity (A), and the environmental information (E) and determines at least one substitutive sense having a high sensory substitution efficiency among the plurality of different substitutive senses as a preferred substitutive sense.

Here, the reactivity is calculated through the reactivity calculator 132, and the activity is calculated through the brain activity calculator 134.

Meanwhile, when the substitution signal is provided to the user so that a plurality of different substitutable senses may be replaced with the substitutive sense by the sensory substitution device 200, activity may be acquired with respect to each individual substitutable sense to calculate a sensory substitution efficiency, and at least one substitutable sense having a low sensory substitution efficiency among the plurality of different substitutable senses may be determined as a reinforceable and substitutable sense.

To provide such a function, the substitutive sense determiner 136 may use a reinforceable and substitutable sense determiner and a first-priority preference sense determiner as shown in the configuration example of FIG. 4. To this end, a substitutable sense information amount analyzer, a preference sense information amount analyzer, and an information amount comparator are additionally used.

First, the adjustment signal transmitter 140 analyzes the amount of information which may be transmitted through each sense.

Then, the substitutable sense information amount analyzer analyzes the amount of information on the substitutable senses in results of the sensory substitution efficiency analyzer 130, and the preference sense information amount analyzer analyzes the amount of information on a first-priority sense in the results of the sensory substitution efficiency analyzer 130.

Subsequently, the information amount comparator determines whether the information amount of the first-priority preference sense is larger than or equal to the information amount of the substitutable sense.

When it is determined that the information amount of the first-priority preference sense is larger than or equal to the information amount of the substitutable sense, the first-priority preference sense determiner makes a decision so that first-priority preference sense information may be transmitted to the sensory substitution device 200.

On the other hand, when it is determined that the information amount of the first-priority preference sense is smaller than the information amount of the substitutable sense, the reinforceable and substitutable sense determiner adds a next-priority sense.

Subsequently, the information amount comparator compares the sum of information amounts of the first-priority sense and the next-priority sense with the information amount of the substitutable sense.

When it is determined that the sum of information amounts of the first-priority sense and the next-priority sense is larger than or equal to the information amount of the substitutable sense, the adjustment signal transmitter 140 transmits a sequence of sense information and a configuration ratio for combining signals.

On the other hand, when it is determined that the sum of information amounts of all preference senses is smaller than the information amount of the substitutable sense, the adjustment signal transmitter 140 requests the sensory substitution device 200 to modify or adjust the resolution of generated information or information to be transmitted.

Meanwhile, a substitutive sense ratio determiner determines a priority order of the plurality of substitution signals provided to the user, the magnitudes of the substitution signals suitable for a user environment, and a component ratio of the sensory substitution signals on the basis of the environmental information collected by the environmental information collector 120 and transmits an adjustment signal to the adjustment signal transmitter 140.

The adjustment signal transmitter 140 provides a correction signal according to the analyzed environmental information-specific sensory substitution efficiencies of the sensory substitution signals to the sensory substitution device 200. In other words, the adjustment signal transmitter 140 transmits the adjustment signal for adjusting the sensory substitution signals according to the analyzed sensory substitution efficiencies of the user to the sensory substitution device 200.

The adjustment signal transmitted through the adjustment signal transmitter 140 may include a signal for adjusting the relative sizes of the plurality of substitution signals provided to the user or the component ratio of the substitution signals on the basis of information on the preferred substitutive sense analyzed by the sensory substitution efficiency analyzer 130, information on the reinforceable and substitutable sense, and the collected environmental information.

According to the exemplary embodiment of the present invention, it is possible to efficiently substitute a user's sense by adjusting a substitution signal provided by a sensory substitution device according to the user's characteristics on the basis of a response of the user whose at least one sense is replaced with one or more other senses using the sensory substitution device.

Meanwhile, the sensory substitution device 200 replaces one of visual, auditory, tactile, taste, and smell information or multisensory information including two or more pieces of different sense information among sense information including visual, auditory, tactile, taste, and smell information with at least one piece of other sense information and transmits the at least one piece of other sense information to the user.

The sensory substitution control device according to the exemplary embodiment of the present invention adjusts a sensory substitution signal provided by the sensory substitution device 200 on the basis of a response or reaction of a user who receives a substitution signal from the sensory substitution device 200 so that the substitution signal may be suitable for the user and learning efficiency may be maximized.

In general, individual users who receive sensory substitution signals may prefer different senses according to sensory organs which are sensory receptors and may react to the received sensory substitution signals to different degrees.

Consequently, it is necessary to adjust even the same sensory substitution signal according to a user who receives the sensory substitution signal and transmit the adjusted sensory substitution signal.

Meanwhile, a user may react to even the same sensory substitution signal to remarkably different degrees according to environments.

For example, a user may make different reactions or responses to audio information replaced by a video signal when the video signal is received in the daytime or a bright interior and when the video signal is received in the night or a dark interior.

Even in the case of a video or audio signal converted into the sense of touch, a user's response or reaction may vary according to whether the user is indoors or outdoors. Consequently, the environmental information collector 120 collects information on the surroundings of the user who receives the sensory substitution signal.

Subsequently, the sensory substitution efficiency analyzer 130 of the sensory substitution control device 100 analyzes sensory substitution efficiency of the user delivered by the sensory substitution device 200 on the basis of a response signal collected by the reaction signal collector 110.

To analyze whether sense information is efficiently replaced with the transmitted sensory substitution signal, the reactivity of a brain region taking charge of a substitutive sense, which is replaced by the sensory substitution device 200, is analyzed with respect to the user who receives the sensory substitution signal. Then, the sensory substitution efficiency may be determined to be higher when the reactivity is higher and may be determined to be lower when the reactivity is lower.

For example, in the case of visual information converted into auditory information, reaction of the auditory cortex and the reactivity of the visual cortex which perceives visual information are analyzed together as a primary reaction to the auditory information transmitted from the brain region of a user who receives the sensory substitution signal so that transmission of the visual information in the form of auditory information may be checked.

Also, the activity of a brain region taking charge of a substitutive sense, which is replaced by the sensory substitution device 200, is checked with respect to the user who receives a substitution signal. Then, the sensory substitution efficiency may be determined to be higher when the activity is higher and may be determined to be lower when the activity is lower.

In the same example as the reactivity, in the case of visual information converted into auditory information, whether the visual information is replaced and transmitted may be checked according to the activity and active location of the visual cortex of the user who receives the sensory substitution signal.

Consequently, the sensory substitution control device according to the exemplary embodiment of the present invention can calculate a sensory substitution efficiency of a user, who receives a substitution signal through the sensory substitution efficiency analyzer 130, on the basis of at least one of reactivity and activity and determine whether the currently provided substitution signal is efficient for the user.

FIG. 2 is a schematic diagram illustrating a sensory substitution control system to which the sensory substitution control device according to the exemplary embodiment of the present invention is applied, and FIG. 3 is an exemplary conceptual diagram of a sensory substitution control process of FIG. 1.

As shown in FIGS. 2 and 3, when the sensory substitution device 200 provides a substitution signal to a user in a plurality of different substitutive senses using activity and reactivity, the sensory substitution control device 100 may acquire reactivity with respect to each individual substitutive sense to calculate a sensory substitution efficiency and may determine at least one substitutive sense having a high sensory substitution efficiency among the plurality of different substitutive senses as a preferred substitutive sense.

Also, when the substitution signal is provided to the user so that a plurality of different substitutable senses may be replaced with the substitutive sense by the sensory substitution device 200, activity may be acquired with respect to each individual substitutable sense to calculate a sensory substitution efficiency, and at least one substitutable sense having a low sensory substitution efficiency among the plurality of different substitutable senses may be determined as a reinforceable and substitutable sense.

The sensory substitution control device 100 determines the priority order of a plurality of substitution signals provided to the user and the relative magnitudes of the substitution signals on the basis of environmental information collected through the environmental information collector 120.

An adjustment signal for adjusting the substitution signals may be transmitted to the sensory substitution device 200 through the adjustment signal transmitter 140 of the sensory substitution control device 100 according to the analyzed sensory substitution efficiency of the user.

The adjustment signal transmitted through the adjustment signal transmitter 140 may include an adjustment signal for adjusting the relative sizes of the plurality of substitution signals provided to the user on the basis of information on the preferred substitutive sense analyzed by the sensory substitution efficiency analyzer 130, information on the reinforceable and substitutable sense, and the collected environmental information.

FIG. 5 is a flowchart illustrating a method of analyzing sensory substitution efficiency in sensory substitution control according to the exemplary embodiment of the present invention.

A sensory substitution adjustment method according to the exemplary embodiment of the present invention will be described below with reference to FIG. 5.

First, the sensory substitution efficiency analyzer detects a synchronization time point at which a sensory substitution signal is started (S510). In the operation S510 of detecting a synchronization time point, when a vision substitution signal is transmitted, a signal synchronizer may detect a transmission time point and synchronize the start point of signal analysis of a brain region with the transmission time point.

Then, a change of a cerebral sensory signal is measured from the synchronization time point (S520).

Subsequently, it is determined whether a received signal is a base signal (S530).

When the received signal is determined to be a base signal (YES) in the operation S530 of determining whether the received signal is a base signal, sense information is stored as basal sense information (S540). A brainwave signal which is collected as soon as the substitution signal is transmitted is converted into a signal required for analysis through necessary preprocessing and sampling processes.

On the other hand, when the received signal is determined not to be a base signal (NO) in the operation S530 of determining whether the received signal is a base signal, reactivity of sensory organs to the signal is analyzed, and sensory reactivity to the sense information stored as the basal sense information is analyzed (S550). In the operation S550 of analyzing the reactivity of the sensory organs to the signal, the efficiency of a received sensory substitution signal is recorded as reactivity which is represented as a result of measuring a change of each individual sense.

To this end, the reaction signal extractor extracts a reaction signal with respect to each individual piece of sense information. As visual information reactivity (Rv), variations of collected brainwave signals of the central occipital channel (Oz) and the right lateral parieto-occipital lobe (PO8) may be used, and as auditory information reactivity (Ra), a change in the right lateral parieto-temporal lobe (TP8) and the right lateral frontal lobe (F8) may be used.

Likewise, for tactile conversion of vision substitution information, the visual information reactivity (Rv) and tactile information reactivity (Rt) may be collected as reaction information using a change in a signal of the cerebral vertex region.

The reactivity calculator 132 first transmits meaningless general information in a signal to be replaced in order to measure variations of substitution signals for each individual sense and stores measured values (Rv, Ra, and Rt) of the corresponding brainwave signals in a base information storage in order to use the measured values as base information.

In the next operation, while a targeted substitution signal is transmitted, reactivity (Rv, Ra, and Rt) of visual, auditory, and tactile information is separately analyzed and compared with the reactivity of each individual sense stored in the base information storage. When the difference is larger, the reactivity is determined to be greater.

In the analysis of sensory signal reactivity, delta waves and theta waves of which the amplitudes are changed relatively easily among brainwave signals are synchronized with a sensory signal to analyze reactivity at a time point which is a certain time after a synchronization signal, and the intensity (variation) of the delta waves and theta waves synchronized and reacting at the synchronization time point on the basis of the base information is used as the reactivity.

Therefore, the reactivity calculator 132 calculates the reaction variation of each individual sensory signal, and calculation of the reaction variation of each individual sense information may involve a frequency converter for analysis, a delta and theta wave extractor, and an individual signal reaction variation calculator. As criteria for high and low reactivity, a high-low value (e.g., double or more) and a threshold value (e.g., 40%) of reactivity may be set on the basis of the base information. When the reactivity is greater than the high-low value, the reactivity may be determined to be high. When the reactivity is greater than the base information by the threshold value or more, the reaction may be determined to be related to a stimulus. When the reactivity is smaller than the base information by the threshold value or more, the reactivity may be determined to be low.

For the reactivity analysis, frequency analysis of brainwave signals, the ERP technique, the ERSP method, etc. may be used.

Subsequently, a sensory substitution efficiency is calculated using the user's reactivity and brain activity and environmental information (S560). The activity of a brain region taking charge of a substitutive sense, which is replaced by the sensory substitution device 200, is checked with respect to the user who receives a substitution signal. Then, the sensory substitution efficiency may be determined to be higher when the activity is higher and may be determined to be lower when the activity is lower.

To this end, the brain action data extractor and the brain activity calculator are used. In the same example as the reactivity, in the case of visual information converted into auditory information, whether the visual information is replaced and transmitted may be checked according to the activity (Av) and active location of the visual cortex of the user who receives the substitution signal. Likewise, the activity (Aa) of the auditory cortex and the activity (At) of the somatosensory cortex related to the sense of touch are checked so that efficiency of each individual substitutable information which is converted from substitution information to activate the brain may be found.

Consequently, the sensory substitution efficiency analyzer may calculate a sensory substitution efficiency of the user who receives the substitution signal on the basis of the reactivity (R) and activity (A) of each individual sensory organ and the environmental information (E) and determine whether the currently provided substitution signal is efficient for the user.

Subsequently, the priority order of preference senses is determined (S570). In other words, finally, the order of senses preferred by the user regarding the currently provided sensory substitution signal is determined on the basis of the analyzed sensory reactivity. With the progress of learning about each individual sense, the sensory substitution efficiency analyzer may explicitly represent the priority order of sense information preferred by the user and whether reactivity is high or low in the way illustrated above.

Therefore, according to the exemplary embodiment of the present invention, when the sensory substitution device 200 provides substitution signals to a user in a plurality of different substitutive senses using such activity and reactivity as shown in FIGS. 4 and 5, the substitutive sense determiner calculates the sensory substitution efficiency (S=R+A+E) of each individual substitutive sense using the reactivity (R), the activity (A), and the environmental information (E) and determines at least one substitutive sense having a high sensory substitution efficiency among the plurality of different substitutive senses as a preferred substitutive sense.

Also, when the substitution signal is provided to the user so that a plurality of different substitutable senses may be replaced with the substitutive sense by the sensory substitution device 200, activity may be acquired with respect to each individual substitutable sense to calculate a sensory substitution efficiency, and at least one substitutable sense having a low sensory substitution efficiency among the plurality of different substitutable senses may be determined as a reinforceable and substitutable sense.

To provide such a function, the substitutive sense determiner may use the reinforceable and substitutable sense determiner and the first-priority preference sense determiner as shown in the configuration example of FIG. 4. To this end, the substitutable sense information amount analyzer, the preference sense information amount analyzer, and the information amount comparator are additionally used.

The substitutive sense ratio determiner determines a priority order of a plurality of substitution signals provided to the user, the magnitudes of the substitution signals suitable for a user environment, and a component ratio of the sensory substitution signals on the basis of the environmental information collected by the environmental information collector and transmits an adjustment signal to the adjustment signal transmitter.

The adjustment signal transmitter transmits an adjustment signal for adjusting the substitution signals to the sensory substitution device 200 according to the analyzed sensory substitution efficiency of the user. The adjustment signal transmitted through the adjustment signal transmitter may include a signal for adjusting the relative sizes of the plurality of substitution signals provided to the user or the component ratio of the substitution signals on the basis of information on the preferred substitutive sense analyzed by the sensory substitution efficiency analyzer, information on the reinforceable and substitutable sense, and the collected environmental information.

FIG. 6 is a flowchart illustrating a method of transmitting an adjustment signal to a sensory substitution device in FIG. 5.

The method of transmitting an adjustment signal to a sensory substitution device will be described with reference to FIG. 6.

First, the adjustment signal transmitter analyzes the amount of information which may be transmitted through each sense, that is, the first information amount of a first-priority sense, which is a first substitutive sense, and the second information amount of a substitutable sense in results of the sensory substitution efficiency analyzer (S610).

Subsequently, it is determined whether the first information amount of the first-priority preference sense is larger than the second information amount of the substitutable sense (S620).

When it is determined in the determination operation S620 that the first information amount of the first-priority preference sense is larger than the second information amount of the substitutable sense (YES), first-priority preference sense information is transmitted (S630).

When it is determined in the determination operation S620 that the first information amount of the first-priority preference sense is smaller than or equal to the second information amount of the substitutable sense (NO), a next-priority sense is added (S640).

Subsequently, the sum of the first information amount of the first-priority preference sense and a third information amount of the next-priority sense is compared with the second information amount of the substitutable sense (S650).

When the sum of the first information amount of the first-priority preference sense and the third information amount of the next-priority sense is determined to be larger than the second information amount of the substitutable sense in the operation S650 of comparing the information amounts (YES), the sequence of sense information and a composition ratio for combining signals are transmitted (S660).

On the other hand, when the sum of all information amounts of preference senses is determined to be smaller than or equal to the second information amount of the substitutable sense in the operation S650 of comparing the information amounts (NO), it is determined whether the sum of information amounts of all preference senses has been compared with the second information amount of the substitutable sense (S670).

When it is determined in the determination operation S670 that the sum of information amounts of all preference senses has been compared with the second information amount of the substitutable sense (YES), the sensory substitution device 200 is requested to modify or adjust the resolution of generated information or information to be transmitted (S680).

When it is determined in the determination operation S670 that the sum of information amounts of all preference senses has not been compared with the second information amount of the substitutable sense (NO), a next-priority substitutive sense is added (S640).

The method of transmitting an adjustment signal according to the exemplary embodiment of FIG. 6 will be described in further detail below.

When the sequence of preference senses is determined through the sensory substitution efficiency analyzer, it is necessary to analyze the amount of information (a bandwidth) which may be transmitted through each sense.

In general, the limited amount of information which may be transmitted through each sensory organ may be found in advance, and the maximum amount of information of each individual sense may be stored in advance for use.

When it is determined in the determination operation S620 that the information amount of the first-priority preference sense is larger than the information amount of the substitutable sense, the adjustment signal transmits only the first-priority sense information.

When the information amount of the first-priority preference sense is smaller than or equal to the information amount of the substitutable sense, the currently transmitted substitutable sense information is not efficiently transmitted to the user. Consequently, it is necessary to additionally transmit second-priority preference sense information in order to ensure a bandwidth for information transmission.

When the sum of information amounts of the first-priority and second-priority preference senses is larger than the information amount of the substitutable sense, a component ratio of signals to be transmitted as a plurality of pieces of sense information is calculated using the sequence of the plurality of pieces of sense information and the information amount of each individual sense, and an adjustment signal including the component ratio of the signals is output.

Meanwhile, when the sum of information amounts of the first-priority and second-priority preference senses is smaller than or equal to the information amount of the substitutable sense, a combination of senses having a larger information amount than the substitutable sense is searched for during a process of adding up to a total of n pieces of preference sensory information.

Even after such a process, the information amount of all the preference senses may be smaller than the information amount of the substitutable sense. In this case, the adjustment signal transmitter transmits a request to change transmission information to the sensory substitution device 200 so that the resolution of generated information or information to be transmitted may be modified or adjusted.

According to the exemplary embodiment of the present invention, it is possible to efficiently replace a user's sense by adjusting a substitution signal provided by a sensory substitution device according to the user's characteristics on the basis of a response of the user whose at least one sense is replaced with one or more other senses by the sensory substitution device.

Each step included in the learning method described above may be implemented as a software module, a hardware module, or a combination thereof, which is executed by a computing device.

Also, an element for performing each step may be respectively implemented as first to two operational logics of a processor.

The software module may be provided in RAM, flash memory, ROM, erasable programmable read only memory (EPROM), electrical erasable programmable read only memory (EEPROM), a register, a hard disk, an attachable/detachable disk, or a storage medium (i.e., a memory and/or a storage) such as CD-ROM.

An exemplary storage medium may be coupled to the processor, and the processor may read out information from the storage medium and may write information in the storage medium. In other embodiments, the storage medium may be provided as one body with the processor.

The processor and the storage medium may be provided in application specific integrated circuit (ASIC). The ASIC may be provided in a user terminal. In other embodiments, the processor and the storage medium may be provided as individual components in a user terminal.

Exemplary methods according to embodiments may be expressed as a series of operation for clarity of description, but such a step does not limit a sequence in which operations are performed. Depending on the case, steps may be performed simultaneously or in different sequences.

In order to implement a method according to embodiments, a disclosed step may additionally include another step, include steps other than some steps, or include another additional step other than some steps.

Various embodiments of the present disclosure do not list all available combinations but are for describing a representative aspect of the present disclosure, and descriptions of various embodiments may be applied independently or may be applied through a combination of two or more.

Moreover, various embodiments of the present disclosure may be implemented with hardware, firmware, software, or a combination thereof. In a case where various embodiments of the present disclosure are implemented with hardware, various embodiments of the present disclosure may be implemented with one or more application specific integrated circuits (ASICs), digital signal processors (DSPs), digital signal processing devices (DSPDs), programmable logic devices (PLDs), field programmable gate arrays (FPGAs), general processors, controllers, microcontrollers, or microprocessors.

The scope of the present disclosure may include software or machine-executable instructions (for example, an operation system (OS), applications, firmware, programs, etc.), which enable operations of a method according to various embodiments to be executed in a device or a computer, and a non-transitory computer-readable medium capable of being executed in a device or a computer each storing the software or the instructions.

A number of exemplary embodiments have been described above. Nevertheless, it will be understood that various modifications may be made. For example, suitable results may be achieved if the described techniques are performed in a different order and/or if components in a described system, architecture, device, or circuit are combined in a different manner and/or replaced or supplemented by other components or their equivalents. Accordingly, other implementations are within the scope of the following claims.

What is claimed is:

1. A sensory substitution control device comprising:
a reaction signal collector configured to collect a user's response or reaction signal to a sensory substitution signal received from a sensory substitution device;
an environmental information collector configured to collect environmental information of the user who receives the sensory substitution signal;
a sensory substitution efficiency analyzer configured to analyze the user's sensory substitution efficiency delivered by the sensory substitution device on the basis of the collected environmental information and generate an adjustment signal for adjusting the sensory substitution signal according to the sensory substitution efficiency; and
an adjustment signal transmitter configured to provide a correction signal according to the analyzed environmental information-specific sensory substitution efficiency of the sensory substitution signal to the sensory substitution device.

2. The sensory substitution control device of claim 1, wherein the reaction signal collector collects a signal obtained from a brain of the user who receives the sensory substitution signal as the response signal.

3. The sensory substitution control device of claim 1, wherein the reaction signal collector comprises a brainwave signal collector configured to collect a brainwave signal from a brain of the user who receives the sensory substitution signal.

4. The sensory substitution control device of claim 1, wherein the reaction signal collector comprises a brain action data collector configured to collect brain action data obtained by measuring a brain of the user who receives the sensory substitution signal.

5. The sensory substitution control device of claim 1, wherein the sensory substitution efficiency analyzer acquires, from the response signal, at least one of:
a reactivity of a brain region taking charge of a substitutive sense of the user, who receives the sensory substitution signal, replaced by the sensory substitution device; and
an activity of a brain region taking charge of a substitutable sense of the user replaced with the substitutive sense by the sensory substitution device, and
the sensory substitution efficiency analyzer calculates the sensory substitution efficiency of the user on the basis of at least one of the reactivity and the activity.

6. The sensory substitution control device of claim 5, wherein when the reactivity is higher, the sensory substitution efficiency is higher, and when the reactivity is lower, the sensory substitution efficiency is lower, and
when the activity is higher, the sensory substitution efficiency is higher, and when the activity is lower, the sensory substitution efficiency is lower.

7. The sensory substitution control device of claim 6, wherein when the sensory substitution device provides the sensory substitution signal to the user in a plurality of different substitutive senses, the sensory substitution efficiency analyzer acquires a reactivity with respect to each individual substitutive sense to calculate a sensory substitution efficiency and determines at least one substitutive sense having a relatively high sensory substitution efficiency among the plurality of different substitutive senses as a preferred substitutive sense, and
the adjustment signal transmitter transmits information on the preferred substitutive sense to the sensory substitution device.

8. The sensory substitution control device of claim 7, wherein when the sensory substitution signal is provided to the user so that a plurality of different substitutable senses are replaced with the substitutive sense by the sensory substitution device, the sensory substitution efficiency analyzer acquires an activity with respect to each individual substitutable sense to calculate a sensory substitution efficiency and determines at least one substitutable sense having a relatively low sensory substitution efficiency among the plurality of different substitutable senses as a reinforceable and substitutable sense, and
the adjustment signal transmitter transmits information on the reinforceable and substitutable sense to the sensory substitution device.

9. The sensory substitution control device of claim 8, wherein when the sensory substitution device provides the sensory substitution signal to the user in a plurality of different substitutive senses, the sensory substitution efficiency analyzer determines a priority order of a plurality of sensory substitution signals provided to the user, magnitudes of the plurality of sensory substitution signals, and a component ratio of the plurality of sensory substitution signals on the basis of the environmental information collected by the environmental information collector, and
the sensory substitution efficiency analyzer generates an adjustment signal for adjusting the relative magnitudes and the component ratio of the plurality of sensory substitution signals provided to the user on the basis of the priority order of the plurality of sensory substitution signals, the relative magnitudes of the plurality of sensory substitution signals, and the component ratio of the plurality of sensory substitution signals and transmits the adjustment signal to the sensory substitution device through the adjustment signal transmitter.

10. The sensory substitution control device of claim 9, wherein the sensory substitution efficiency analyzer comprises:
an information amount analyzer configured to analyze the amount of information transmitted to the user regarding each of the plurality of substitutive senses and the plurality of substitutable senses on the basis of the response signal and the environmental information;
a first-priority preference sense determiner configured to determine a first-priority preference sense among the plurality of substitutive senses according to the priority order;
an information amount comparator configured to compare the information amount analyzed regarding the first-priority preference sense with the information amount analyzed regarding the substitutable sense and compare a sum of the information amount regarding the first-priority preference sense and the information amount regarding a next-priority substitutive sense, which has a lower priority order than the first-priority preference sense, with the information amount regarding the substitutable sense when the information amount regarding the first-priority preference sense is smaller than the information amount regarding the substitutable sense; and
a substitutive sense ratio determiner configured to determine magnitudes and a component ratio of a first sensory substitution signal for the first-priority preference sense and a second sensory substitution signal for the next-priority substitutive sense according to a comparison result of the information amount comparator.

11. The sensory substitution control device of claim 10, wherein when the information amount regarding the first-priority preference sense is larger than the information amount regarding the substitutable sense, the substitutive sense ratio determiner transmits a first adjustment signal to the sensory substitution device through the adjustment signal transmitter so that the first sensory substitution signal for the first-priority preference sense is transmitted to the user,
when the information amount regarding the first-priority preference sense is smaller than or equal to the information amount regarding the substitutable sense, the substitutive sense ratio determiner transmits a second adjustment signal to the sensory substitution device through the adjustment signal transmitter so that the second sensory substitution signal for the next-priority substitutive sense is transmitted to the user together with the first sensory substitution signal, and
when a sum of all information amounts analyzed regarding the plurality of substitutive senses is smaller than the information amount regarding the substitutable sense, the substitutive sense ratio determiner requests the sensory substitution device to modify or adjust a resolution of generated information or information to be transmitted.

12. The sensory substitution control device of claim 9, wherein the sensory substitution efficiency analyzer detects a synchronization time point at which a sensory substitution signal is started and measures a change in a cerebral sensory signal from the synchronization time point,
the sensory substitution efficiency analyzer determines whether a received signal is a base signal, stores sense information as the basal sense information when the received signal is a base signal, and analyzes reactivity of sensory organs with respect to the received signal when the received signal is not a base signal, and
the sensory substitution efficiency analyzer determines reactivity to the substitutive sense according to a difference between reactivity of each individual sensory organ and reactivity of each individual sense stored in a base information storage and determines a priority order of preference senses.

13. The sensory substitution control device of claim 1, wherein the sensory substitution efficiency analyzer comprises:
a reaction signal extractor configured to extract brain reaction signals including a central occipital signal and a right lateral parieto-occipital lobe signal related to a visual information reaction, a right lateral parieto-temporal lobe signal and a right lateral frontal lobe signal related to an auditory information reaction, and a cerebral vertex signal related to a tactile information reaction among brainwave signals collected by the reaction signal collector;
a reactivity calculator configured to generate frequency-domain brainwave data by converting frequencies of the brain reaction signals extracted by the reaction signal extractor, extract delta wave and theta wave data from the frequency-domain brainwave data, and calculate a reaction variation of each substitutive sense by comparing the delta wave and theta wave data with base state data;
a brain action data extractor configured to extract substitutive sense-related brain action data of a visual cortex region, an auditory cortex region, and a somatosensory cortex region from brain action data collected by the reaction signal collector;
a brain activity calculator configured to calculate substitutive sense-related brain activity including visual cortex activity, auditory cortex activity, and somatosensory cortex activity on the basis of the substitutive sense-related brain action data;
a learning part configured to generate a substitutive sense adjustment model for adjusting the sensory substitution signal using the environmental information, the reaction variation of each substitutive sense, and the substitutive sense-related brain activity on the basis of learning; and
a substitutive sense determiner configured to generate an adjustment signal for adjusting the sensory substitution signal on the basis of the substitutive sense adjustment model.

14. A sensory substitution control method comprising:
detecting, by a sensory substitution efficiency analyzer, a synchronization time point at which a sensory substitution signal is started;
measuring a change in a cerebral sensory signal from the synchronization time point;
determining whether a received signal is a base signal;
when it is determined that the received signal is a base signal, storing sense information as base sense information;
when it is determined that the received signal is not a base signal, analyzing reactivity of sensory organs with respect to the received signal;
analyzing sensory reactivity to the sense information stored as the base sense information;
calculating a sensory substitution efficiency using a user's reactivity and brain activity and environmental information; and
determining a priority order of preference senses.

15. The sensory substitution control method of claim 14, wherein the analyzing of the reactivity of the sensory organs comprises:
extracting, by the sensory substitution efficiency analyzer, brain reaction signals including a central occipital signal and a right lateral parieto-occipital lobe signal related to a visual information reaction, a right lateral parieto-temporal lobe signal and a right lateral frontal lobe signal related to an auditory information reaction, and a cerebral vertex signal related to a tactile information reaction among brainwave signals collected by a reaction signal collector;
converting, by the sensory substitution efficiency analyzer, frequencies of the brain reaction signals extracted by a reaction signal extractor to generate frequency-domain brainwave data, extracting delta wave and theta wave data from the frequency-domain brainwave data, and comparing the delta wave and theta wave data with base state data to calculate a reaction variation of each substitutive sense;
extracting, by the sensory substitution efficiency analyzer, substitutive sense-related brain action data of a visual cortex region, an auditory cortex region, and a somatosensory cortex region from brain action data collected by the reaction signal collector;
calculating, by the sensory substitution efficiency analyzer, substitutive sense-related brain activity including visual cortex activity, auditory cortex activity, and somatosensory cortex activity on the basis of the substitutive sense-related brain action data;
generating, by the sensory substitution efficiency analyzer, a substitutive sense adjustment model for adjusting the sensory substitution signal using the environmental information, the reaction variation of each substitutive sense, and the substitutive sense-related brain activity on the basis of learning; and
generating, by the sensory substitution efficiency analyzer, an adjustment signal for adjusting the sensory substitution signal on the basis of the substitutive sense adjustment model.

16. The sensory substitution control method of claim 15, wherein the reactivity is set on the basis of base information through one of a high-low value and a threshold value of reactivity.

17. The sensory substitution control method of claim 14, wherein the analyzing of the sensory reactivity comprises analyzing the sensory reactivity through one method among frequency analysis of brainwave signals, an event-related potential (ERP) technique, and an event-related spectral perturbation (ERSP) method.

18. The sensory substitution control method of claim 14, wherein the calculating of the sensory substitution efficiency comprises:
calculating, by the sensory substitution efficiency analyzer, a sensory substitution efficiency on the basis of reactivity of each individual sensory organ of the user who receives the sensory substitution signal, activity of a brain region taking charge of a substitutable sense, which is replaced by the sensory substitution device, with respect to the user who receives the sensory substitution signal, and the environmental information; and
determining the sensory substitution efficiency to be higher when the activity is higher and determining the sensory substitution efficiency to be lower when the activity is lower.

19. A sensory substitution control method comprising:
analyzing, by an adjustment signal transmitter, the amount of information transmissible through each sense, that is, an information amount of a first-priority preference sense, which is a first substitutive sense, and an information amount of a substitutable sense in results of a sensory substitution efficiency analyzer;
determining whether the information amount of the first-priority preference sense is larger than the information amount of the substitutable sense; and
when it is determined that the information amount of the first-priority preference sense is larger than the information amount of the substitutable sense, transmitting first-priority preference sense information.

20. The sensory substitution control method of claim 19, further comprising:
when it is determined that the information amount of the first-priority preference sense is smaller than or equal to the information amount of the substitutable sense, adding a next-priority sense;
comparing a sum of the information amount of the first-priority preference sense and information amounts of next-priority senses with the information amount of the substitutable sense;
when it is determined that the sum of the information amount of the first-priority preference sense and the information amounts of the next-priority senses is larger than the information amount of the substitutable sense, transmitting a sequence of sense information and a composition ratio for combining signals;
when it is determined that even a sum of all information amounts of preference senses is smaller than the information amount of the substitutable sense, determining whether a sum of information amounts of all preference senses has been compared with the information amount of the substitutable sense;
when it is determined that the sum of the information amounts of all preference senses has been compared with the information amount of the substitutable sense, requesting a sensory substitution device to modify or adjust a resolution of generated information or information to transmitted; and
when it is determined that the sum of the information amounts of all the preference senses has not been compared with the information amount of the substitutable sense, adding a next-priority sense.
